(19) United States
(12) Patent Application Publication
(10) Pub. No.: US 2010/0028360 A1
(43) Pub. Date: Feb. 4, 2010

(54) METHODS FOR THE MODULATION OF BRAIN PROGESTAGEN SIGNALING IN THE PREVENTION AND TREATMENT OF NEUROLOGICAL DISORDERS AND NEURODEGENERATIVE DISEASES

(76) Inventor: Craig Stephen Atwood, Madison, WI (US)

Correspondence Address: CRAIG S. ATWOOD, PH.D., 1125 RED TAIL DR., VERONA, WI, 53593, US

(21) Appl. No.: 12/508,676

(22) Filed: Jul. 24, 2009

Related U.S. Application Data
(60) Provisional application No. 61/083,918, filed on Jul. 26, 2008.

(51) Int. Cl. A61K 39/395; A61K 31/56; A61K 38/16; A61K 38/08; A61K 31/7052; C12N 5/00; C12N 5/0735; A61P 25/00

(52) U.S. Cl. 424/158.1; 514/177; 514/170; 514/12; 514/15; 514/44 A; 435/377; 435/366

(57) ABSTRACT

The present invention relates to methods for modulating progestagen signaling for treating neurological disorders or neurodegenerative disease, or preventing or delaying its onset in individuals deemed by competent observation and testing to be susceptible thereto. Progestagens can be administered to elevate serum and brain levels of progestagens and induce neurogenesis. Progestagen therapy may prevent some of the neurodegenerative and cognitive changes associated with developmental and aging associated neurological disorders and neurodegenerative diseases. Progestagen therapy together with suppression of GnRH, kisspeptin, LH and/or FSH signaling also may be used for treating neurological disorders or neurodegenerative diseases. The invention also relates to methods for inhibiting or delaying blastulation during embryogenesis, and neurogenesis during embryogenesis, fetal, neonatal, childhood, puberty or adult life. Blocking progestagen, estrogen and/or opioid signaling with receptor antagonists will inhibit neurogenesis. The invention also relates to using progestagens in vitro to induce neurogenesis in embryonic or adult stem cells.

### CROSS-REFERENCE TO RELATED APPLICATIONS

The present application claims the benefit under 35 U.S.C. §119(e) to U.S. Provisional Patent Application No. 61083918, filed on Jul. 26, 2008, the content of which is incorporated herein by reference in its entirety.

### SUMMARY OF THE INVENTION

The inventors have shown that progesterone is required for the differentiation of hESC into NPC and neuronal cells. Progesterone-induced neurogenesis has many potential uses. Also, GnRH signaling is required for normal neuronal transmission.

It has been discovered that progesterone promotes hESC differentiation toward a neuroectodermal phenotype since 1). P4 induces nestin expression in hESC (FIG. 1b), 2). P4 withdrawal from embryoid bodies (EB) inhibited neuroectodermal rosette and therefore NPC formation (FIG. 1c(ii)), 3). the PR antagonist, RU486, completely blocked both EB and neuroectodermal rosette/NPC formation as demonstrated morphologically (no rosette development, decreased structure size; FIG. 1c(iii-iv)) and biochemically (suppression of nestin expression; FIG. 1d), 4). Inhibition of ER signaling, required for PR expression, with the ER antagonist ICI 174 864 also blocked both EB and neuroectodermal rosette formation (FIG. 1c(iv) and 1d), and 5). Human chorionic gonadotropin (hCG) rapidly upregulated cholesterol transport by steroidogenic acute regulatory protein (StAR) and progesterone synthesis and secretion by hESC (FIG. 2). These data indicate hCG has a dual role during early human embryonic development, promoting cell proliferation and neural induction via upregulation of StAR mediated cholesterol transport for steroidogenesis.

According to this invention, elevating the levels of brain progestagens, either by administration of exogenous progestagens or by hCG-induced progestagen production in vivo, promotes neurogenesis and inhibits the progression of a neurological disorder or a neurodegenerative disease in a person suffering therefrom, and consequently can be used to treat the disease.

According to this invention, elevating the levels of brain progestagens halts neuronal and synaptic loss and inhibits the progression of a neurological disorder or a neurodegenerative disease in a person suffering therefrom, and consequently can be used to treat the disease.

Further, according to this invention, maintaining high progestagen levels comparable to pregnancy levels (10-300 ng/mL) inhibits the development of neurological disorders and neurodegenerative diseases in a person susceptible to the disease. Thus, in accordance with this discovery, neurological disorders and neurodegenerative diseases can be prevented in an individual by maintaining progestagens at levels that are sufficiently high to be effective for such prevention.

As another embodiment of these findings, progestagens can be used to differentiate hESC into NPC in vitro and in vivo. Media containing essential nutrients and including progestagens can be used for example to differentiate embryonic stem cells into neuronal cell types.

As another embodiment of these findings, antagonists of PR signaling such as RU-486 can be used to inhibit blastulation during embryogenesis is described.

As another embodiment of these findings, antagonists of ER signaling such as ICI 182,780 can be used to inhibit blastulation during embryogenesis is described.

As another embodiment of these findings, antagonists of OR signaling such as ICI 174,864 can be used to inhibit blastulation during embryogenesis is described.

As another embodiment of these findings, antagonists of PR signaling such as RU-486 can be used to inhibit neurogenesis during embryogenesis, fetal, neonatal, childhood, puberty or adult life is described.

As another embodiment of these findings, antagonists of ER signaling such as ICI 182,780 can be used to inhibit neurogenesis embryogenesis, fetal, neonatal, childhood, puberty or adult life is described.

As another embodiment of these findings, antagonists of OR signaling such as ICI 174,864 can be used to inhibit neurogenesis embryogenesis, fetal, neonatal, childhood, puberty or adult life is described.

As another embodiment of these findings, antagonists of OR signaling such as ICI 174,864 can be used to inhibit neurogenesis embryogenesis, fetal, neonatal, childhood, puberty or adult life is described.

According to this invention, modulating blood serum and brain levels of one or more of kisspeptin, GnRH, FSH and LH halts neuronal and synaptic loss and inhibits the progression of a neurological disorder or a neurodegenerative disease in a person suffering therefrom, and consequently can be used to treat the disease.

As another embodiment of these findings, agonists and antagonists of kisspeptin, GnRH, FSH and LH such as leuprolide, buserelin nafarelin deslorelin histrelin goserelin triptorelin abarelix cetrorelix ganirelix acyline can be used to promote neuronal transmission and neurogenesis and inhibits the progression of a neurological disorder or a neurodegenerative disease in a person suffering therefrom, and consequently can be used to treat the disease.

Thus, the invention entails a method of treating neurological disorders and neurodegenerative diseases in a person suffering therefrom and a method of preventing a neurological disorder or neurodegenerative disease in a person susceptible thereto by administration to the person of an neurological disorder/ neurodegenerative disease-treatment-effective amount or a neurological disorders/neurodegenerative disease-prevention-effective amount, respectively, of a compound or combinations which will maintain or elevate the level in the person (e.g., the level in the person's serum or brain) of a hormone selected from the group consisting of progesterone, pregnenolone, 17α-hydroxypregnenolone, 17α-hydroxyprogesterone, allopregnenolone, alloprogesterone, levonorgestrel, medroxyprogesterone, etonogestrel or other progesterone analogs). Among such compounds are those selected from the group consisting of progestagens and physiologically acceptable salts thereof. The method also entails administering agonists and antagonists of kisspeptin, GnRH, FSH and LH together with progestagens to promote neurogenesis and neuronal transmission.

The invention also entails a method for blocking progestagen, estrogen and opioid signaling using PR, ER and OR antagonists in an embryo, fetus, neonate, child, adolescent or adult person by administration to said life form an agent selected from the group consisting RU-486, ICI 182,780, ICI 174,864 or other ER, PR or OR antagonists. The invention also entails a method for inducing neurogenesis in vitro where embryonic or adult stem cells are grown in media containing essential nutrients plus progestagen.

### DESCRIPTION

The present invention concerns methods for preventing or delaying the onset of a neurological disorder or neurodegenerative disease in persons susceptible to the disorder/disease and for treating or slowing the development of the disorder/disease in persons suffering therefrom. The present invention also concerns methods for inhibiting neurogenesis. The present invention also concerns methods for inducing neurogenesis in vitro from embryonic stem cells.

### BACKGROUND OF THE INVENTION

Global deterioration of cognitive, motor, sensory and vascular functioning in the brain occurs in all individuals with age. While this decline is pervasive, the rates at which these degenerative changes occur, and the brain structures involved, are variable. This variability, which is defined by genetic and environmental factors, determines who will experience normal changes in brain function versus those who will develop age-related central nervous system diseases.

One of the major changes is the loss of cognitive function. For some cognitive abilities such as episodic memory, short-term (working) memory and fluid intelligence, the onset of decline occurs relatively early and continues into late life (Bäckman et al., 1999). From the mid-seventies of life and onward the magnitude of age-related decline appears to be quite similar across most forms of memory and cognition. The timing and progress of such changes is variable, but eventually presents clinically as dementia (e.g. Alzheimer's disease). A second form of degenerative decline involves those centers of the brain that control motor function and that leads to the loss of motor coordination and diseases such as Parkinson's disease. A third form of degeneration involves the cerebrovasculature that provides nourishment to the brain. Numerous changes related to cerebral blood flow are associated with the aging cerebrovasculature and severe changes present as either hemorrhagic, or ischemic stroke.

### Cognitive Diseases

Alzheimer's disease (AD) is the term used to describe one of several dementing disorders, brain diseases that progressively lead to loss of mental and physical functions, regardless of the age of onset. Of the dementias, AD is the most prevalent afflicting ˜4.5 million Americans.

AD is the fourth leading cause of death in the United States and in 2000 affected 4.5 million Americans (Hebert et al., 2003). Estimates indicate that ˜3% of Americans between ages 65 and 74, 19% between the ages 75-84, and 47% over the age of 85 develop the disease (Evans et al., 1989) and that ˜60% of nursing home patients over age 65 suffer from AD. Clinically AD is characterized by a progressive decline in multiple cognitive functions and begins with amnestic mild cognitive impairment (MCI), a transition between normal aging and dementia. Current data suggest that conversion from MCI to dementia occurs at a rate of 10 to 15% per year (Petersen and Morris, 2003) with ˜80% conversion by the sixth year of follow-up. Progression from MCI leads to early AD (EAD) which is clinically characterized by a) a decline in cognitive function from a previous higher level, b). decline in one or more areas of cognition in addition to memory, c) a clinical dementia rating scale score of 0.5 to 1, d) impaired ADLs, and e) a clinical evaluation that excludes other causes of dementia. The disease eventually progresses to late stage AD (LAD) characterized by severe dementia with disorientation, global cognitive deficits, profound memory impairment, inattention, altered personality, difficulty speaking and comprehending, and impaired gait and movement.

The annual cost of caring for individuals with AD in institutional and community settings in the U.S. is about $100 billion for direct costs alone. As the population ages, the number of AD patients and the costs of their care will rise as well. Without preventive strategies, there may be 14 million Americans with AD by the year 2040 (Hebert et al., 2003).

AD can be diagnosed postmortem from microscopic abnormalities found in brain tissue. See “Consensus Recommendations for the Postmortem Diagnosis of Alzheimer's Disease,” in Neurobiology of Aging (in the press), pre-published on the World-wide Web at the site for the Alzheimer Research Forum at http://www.alzforum.org/members/forums/consensus/index.html. The five principal overt neuropathological abnormalities are 1) loss of neurons, 2) loss of synaptic connections, 3) senile or neuritic plaques (chemical deposits consisting of degenerating nerve cells combined with a form of protein called beta amyloid), 4) neurofibrillary tangles (malformations within nerve cells) and 5) microgliosis. The brains of AD patients usually contain all these abnormalities on autopsy examination, particularly in the hippocampus, amygdala, entorhinal cortex, and neocortex. NFT are composed of intracellular deposits of paired helical filaments composed of hyperphosphorylated tau. Senile plaques are present in two forms: a) diffuse plaques (DP) composed of amorphous extracellular deposits of Aβ that lack neurites, and b) neuritic plaques (NP) composed of extracellular deposits of insoluble Aβ surrounded by dystrophic neurites, reactive astrocytes, and activated microglia. Recent studies suggest that, in addition to insoluble Aβ present in SP, soluble Aβ oligomers are present in the AD brain and may represent the main toxic form of Aβ, thus implicating them in the disease process (Glabe et al., 2006; Klein et al., 2004).

### Motor Coordination Diseases

Parkinson's disease (PD) is a progressive neurological disorder characterized by rest tremor, bradykinesia, rigidity and postural instability and afflicts˜1-1.5 million Americans. Parkinson disease affects movement (motor symptoms). Typical other symptoms include disorders of mood, behavior, thinking, and sensation (non-motor symptoms). PD is neuropathologically characterized by the progressive loss of neurons in the substantia nigra pars compacta (SNpc) and other subcortical nuclei. This neuronal population is associated with intracytoplasmic Lewy bodies (composed primarily of □-synuclein and highly ubiquitinated proteins), and dystrophic (Lewy) neurites mainly in subcortical nuclei and hippocampus and, less frequently in the cerebral cortex.

There are other disorders that are called Parkinson-plus diseases. These include: multiple system atrophy (MSA), progressive supranuclear palsy (PSP) and corticobasal degeneration (CBD). Some include dementia with Lewy bodies (DLB)—while idiopathic Parkinson's disease patients also have Lewy bodies in their brain tissue, the distribution is denser and more widespread in DLB. Even so, the relationship between PD, PD with dementia (PDD), and dementia with Lewy bodies (DLB) might be most accurately conceptualized as a spectrum, with a discrete area of overlap between each of the three disorders. The natural history and role of Lewy bodies is little understood. Wilson's disease (hereditary copper accumulation) may present with parkinsonistic features.

These Parkinson-plus diseases may progress more quickly than typical idiopathic Parkinson disease. If cognitive dysfunction occurs before or very early in the course of the movement disorder then DLBD may be suspected. Early postural instability with minimal tremor especially in the context of ophthalmoparesis should suggest PSP. Early autonomic dysfunction including erectile dysfunction and syncope may suggest MSA. The presence of extreme asymmetry with patchy cortical cognitive defects such dysphasia and apraxias especially with “alien limb” phenomena should suggest CBD.

### Cerebrovascular Diseases

Changes in the aging cerebrovasculature include decreased microvascular density, loss of endothelium, increased tortuosity, twisted/string vessels, fragmentation of the microvasculature, loss of the fine perivascular neural plexus and lumpy vessels. Such changes are far more pronounced in AD (e.g. Kalaria, 1996). Cerebral capillary distortions caused by these changes create ‘disturbed’ rather than ‘laminar’ blood flow that impair normal delivery of essential nutrients to brain neurons as well as impede catabolic outflow of waste products (de la Torre et al., 1997).

Severe changes result in stroke, the major cerebrovascular disease. Every year 750,000 Americans suffer from a new or recurrent stroke which is the third leading cause of death killing nearly 160,000 Americans/year. Stroke results when blood flow to an area of the brain is interrupted (e.g. by either a blood clot; ischemic stroke), or by a vessel rupture (hemorrhagic stroke). Neuronal cells in the immediate vicinity of the lesion (infarct) die within minutes and in the hours during and after the stroke, the area surrounding the infarct (penumbra) starts releasing chemicals that set off a chain reaction called the ‘ischemic cascade’. This chain reaction, if untreated, leads to the death of neuronal cells in a larger area of brain tissue for which the blood supply is compromised but not completely cut off.

### Traumatic Brain Injury

Traumatic injuries to the brain, also called intracranial injury, or simply head injury, occurs when physical trauma causes brain damage. Traumatic brain injury (TBI) can result from a closed head injury or a penetrating head injury and is one of two subsets of acquired brain injury (ABI). The other subset is non-traumatic brain injury, or injuries that do not involve external mechanical force (e.g. stroke, meningitis, anoxia). Parts of the brain that can be damaged include the cerebral hemispheres, cerebellum, and brain stem.

TBI can result from transportation accidents involving automobiles, motorcycles, bicycles, and pedestrians (and cause half of all TBIs and the largest portion of TBI in people under age 75; (Traumatic Brain Injury: Hope Through Research. NINDS, 2002). For those aged 75 and older, falls cause the majority of TBIs (Traumatic Brain Injury: Hope Through Research. NINDS, 2002). Approximately 20% of TBIs are due to violence, such as firearm assaults and child abuse, and about 3% are due to sports injuries (Traumatic Brain Injury: Hope Through Research. NINDS, 2002). Half of TBI incidents involve alcohol use (Traumatic Brain Injury: Hope Through Research. NINDS, 2002). Traumatic brain injury is a frequent cause of major long-term disability in individuals surviving head injuries sustained in war zones (Hoge et al., 2008).

### Neurological Disease Etiology

The underlying neurodegenerative mechanisms leading to these major age-related declines in brain function are the focus of intense research. Naturally, most research in the area of age-related neurodegeneration is disease centred and has predominantly focused on the overt neuropathological changes unique to each disease. Since all individuals experience some decrease in brain function during the period of advanced age, age-related ‘diseases’ of the brain may represent nothing more than progression along a continuum. This is supported by epidemiological studies that have identified numerous, diverse risk factors for these diseases, however the risk factor common to all these diseases is increased age. In this connection, multiple cognitive, neuropathological and biochemical changes are often seen in a particular disease. For example, PD involves the loss of both motor and non-motor (cognitive) function while diffuse Lewy bodies are often detected in individuals diagnosed with AD. Taken together these observations raise the possibility that the disease specific neuropathological changes (neuron loss, amyloid deposits, Lewy bodies, stroke, etc) are triggered by a common age-related mechanism. And the development of a particular disease state is dependent upon the individual's unique genetic profile and environmental exposures.

Epidemiological and biochemical evidence suggests that post-reproductive changes in hypothalamic-pituitary-gonadal (HPG) axis hormones may be the common mechanism driving degenerative changes in the aging brain (Bowen and Atwood, 2004). These changes are driven by an abrupt decline in the secretion of gonadal estrogens during menopause, and similarly, a gradual and progressive decline in gonadal androgens secretion during andropause. As a result, much research has focused on understanding how the sex steroids may modulate brain aging. Both estrogen and testosterone production are under the control of the HPG axis, which stimulates sex steroid production by increasing the secretion of gonadotropin releasing hormone (GnRH) from the hypothalamus, which in turn stimulates the anterior pituitary to secrete gonadotropin, luteinizing hormone (LH) and follicle stimulating hormone (FSH). A negative feedback mechanism between gonadotropin and gonadal steroid production is mediated by the inhibition of hypothalamic GnRH secretion that modulates the levels of these circulating hormones. As a result of the changes in serum concentrations of sex steroids following menopause/andropause, the other hormones of the HPG axis become dysregulated owing to the complex feedback loops that normally keep these hormones in balance. For example, the loss of negative feedback by estrogen on gonadotropin production following menopause results in a three- to four-fold and a four- to eighteen-fold increase in the concentrations of serum LH and FSH, respectively (see Bowen et al., 2002 and references therein). Likewise, similar changes are observed in men. The concentration of inhibins, responsible for modulating FSH production, and activins also are altered. Importantly, receptors for all these hormones are expressed in the brain (Vadakkadath Meethal and Atwood, 2005). Since the brain is not normally exposed to post-menopausal/andropausal concentrations of these HPG axis hormones, it has been suggested that this might be a mechanism by which (brain) aging is driven (Bowen et al., 2002; Bowen and Atwood, 2004).

Evidence to support altered HPG hormone signaling as promoting neurodegeneration includes findings that the increase in serum gonadotropins with the loss of sex steroids increases the processing of the amyloid-beta precursor protein (AβPP) towards the amyloidogenic pathway in neuroblastoma cells, leading to decreased secretion of AβPP, increased AβPPCT100 production and increased generation and secretion of Aβ, the major component of amyloid deposits in AD (Bowen et al., 2004). Moreover, suppression of serum gonadotropins with GnRH agonists results in suppression of total brain Aβ1-42 and Aβ1-40 concentrations by 3.5-fold and 1.5-fold, respectively, after 2 months of treatment in C57/B16 mice (Bowen et al., 2004). In addition, in a mouse model of amyloidosis (Tg2576 mice carrying the Swedish AβPP mutation) the GnRH agonist leuprolide acetate halts Aβ deposition in aged transgenic mice and improves cognitive performance (Casadesus et al., 2006a). LH also dose-dependently alters tau phosphorylation, increases oxidative stress and induces cytotoxicity in neuroblastoma cells (Casadesus et al., 2006b). Cognitive decline also is halted in AβPP-transgenic mice receiving GnRH agonists (Casadesus et al., 2007).

Further evidence to support the loss of brain sex steroids as an etiological factor in the development of neurological disorders or neurodegenerative diseases is evidenced by the finding that progesterone promotes neurogenesis, where neurogenesis is defined as the concomitant proliferation and differentiation of human embryonic stem cells (hESC) into neural stem cells/neural precursor cells (NPC), or neural stem cells into neuronal cell types. hESC treated with progesterone (P4) and estradiol (E2), which is known to upregulate P4 receptor (PR) expression significantly decreased the rate of hESC proliferation 29%, 16% and 23%, respectively, compared to untreated control cells (FIG. 1a), suggesting these sex steroids may be differentiating hESC. A screen of germline markers indicated P4 increased the expression of the neuroectodermal marker nestin, an early marker of neural precursor cells (NPC; FIG. 1b). E2 also induced nestin expression (205- and 220-kDa variants), albeit at a lower level, perhaps as a result of E2-induced P4 receptor (PR) expression (Levine et al., 1985) and PR signaling from endogenous P4 production.

Typically, in the presence of P4, control rosettes display a minimum of three rosette structures inside of the cystic cavities of the hESC structures (FIG. 1c(i)). hESC grown in specially made media containing all the usual neural induction components with the exception of P4 did not form neuroectodermal rosettes inside the cystic cavities of the structures (FIG. 1c(ii)). To confirm the requirement for sex steroids in the induction of NPC, hESC were treated with a PR antagonist (RU-486) or a estrogen receptor (ER) antagonist (ICI 182,780) just prior to entering the EB stage. No rosettes were detected in any structure treated with RU-486 or ICI 182,780 (FIG. 1c(iii)). The absence of nestin in PR- and ER-antagonist treated pre-EB structures (FIG. 1d) confirmed the requirement for P4 and ER signaling for NPC formation. These results indicate that P4 and E2 are essential for the concomitant proliferation and differentiation of hESC into NPC. Removal of progesterone from hESC or EBs, or addition of PR, ER and/or OR antagonists to hESC or EBs, prevents neurogenesis.

To understand how GnRH1 and GnRH1 analogues affect neuronal excitability in young and old brain, we performed electrophysiological studies on isolated rat hippocampal slices. The superfusion of human GnRH1 evoked a 22% increase in neuronal excitability in hippocampal slices from young (37 day old) rats compared to controls (FIG. 3A, C). In contrast, the same concentration of GnRH1 evoked a 50% increase in the maximum amplitude of population spikes in hippocampal slices from old (19 month old) rats compared to controls (FIG. 4B, C). The GnRH1-induced changes in field responses from the cell body layer of the CA1 in young rats was not significant (maximum amplitude from control is 7.0±1.4mV, while the GnRH1 treated is 7.9±2.7 mV, n=5, p=0.548; FIG. 4C). However there was a significant increase in field responses from the cell body layer of the CA1 of the hippocampal slices from old rats when superfused with the same concentration of GnRH1 (50 nM) (2.54±0.18 mV to 3.99±0.84 mV (n=3, p=0.05; FIG. 4C). These results indicate that GnRH1 can modulate neuronal excitability in aged rat hippocampal slices and suggests that GnRH1 signaling via the hippocampal GnRHR1 may be an important modulator of impulse transmission in the aging brain. The significantly higher effect of GnRH1 on neuronal excitability in old brain hippocampus compared to young brain is very interesting given the fact that the level of neuronal excitability is much lower in slices from old rat (the maximum population spike amplitude is less then half in old rat hippocampus compared to young (7.0±1.39 mV in young as against 2.5±0.18 mV in old, n=3, p<0.05) and that the GnRH1 secretion is elevated during aging. These results demonstrate that GnRH can increase neuronal excitability in hippocampal slices from old rat compared to young rat indicating a role for GnRH signaling in the development and maintenance of brain function.

### BRIEF DESCRIPTION OF THE DRAWINGS

FIG. 1 illustrates the effects of progesterone and estrogen on neurogenesis.

FIG. 2 illustrates the effects of hCG on progesterone production by hESC.

FIG. 3 illustrates the effect of GnRH1 on neuronal excitability in hippocampal slices from old rats.

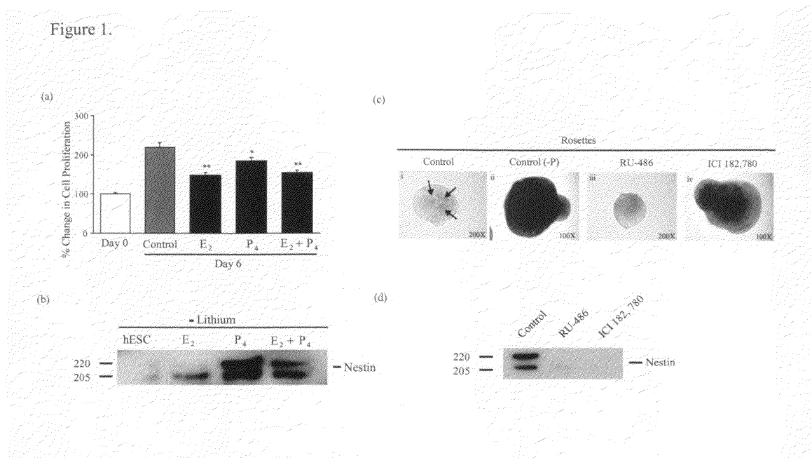

FIG. 1. P4 differentiates hESC into neuroectoderm. (a) hESC were grown in TESR1 media—Li in the presence of E2 (10 nM), P4 (2 μM) or E2 (10 nM)+P4 (2 μM) for 6 d and cell proliferation measured using the trypan blue assay. Results are expressed as mean±SEM, n=3 (*p<0.05, **p<0.005 compared to 6 d control). (b) Equal amounts of protein from cell lysates of hESC treated for 9 d as described above were analyzed by immunoblot using a monoclonal antibody against nestin (clone 10C2; Chemicon, Calif., USA). (c) hESC were cultured for 4 d, enzymatically lifted and placed into EB media (containing serum) and rocked gently for an additional 4 d prior to being placed in one of two neural induction medias for an additional 11 days, where P4 was either absence or present. During this transition from EB to rosettes, colony structures treated with P4 also were treated with a PR antagonist (RU-486; 20 μM) or ER antagonist (ICI 182,780) for 11 d. At 19 d, morphological observation and molecular analysis were performed. (i) Control structure, a minimum of three rosette structures were observed inside the cystic cavity (arrows). (ii) hESC structure in the absence of P4. (iii) PR antagonist treated rosettes (RU-486, 20 μM). (iv) ER antagonist (ICI 182,780). (d) Equal amounts of protein from cell lysates of the above structures were analyzed by immunoblot with the monoclonal antibody against human nestin.

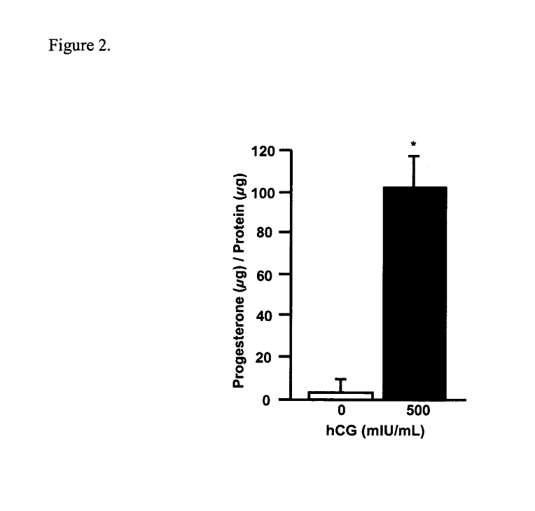

FIG. 2. hCG induces P4 synthesis and secretion from hESC. hESC were treated with hCG (500 mIU/mL) in TESR1 media each day for 6 d, the media collected and pooled each day (15 mL total), lyophilized and resuspended in 2 mL of TESR1 media for ELISA of P4. Results are expressed as ug P4/ug cellular protein (mean±SEM, n=3, *p<0.001).

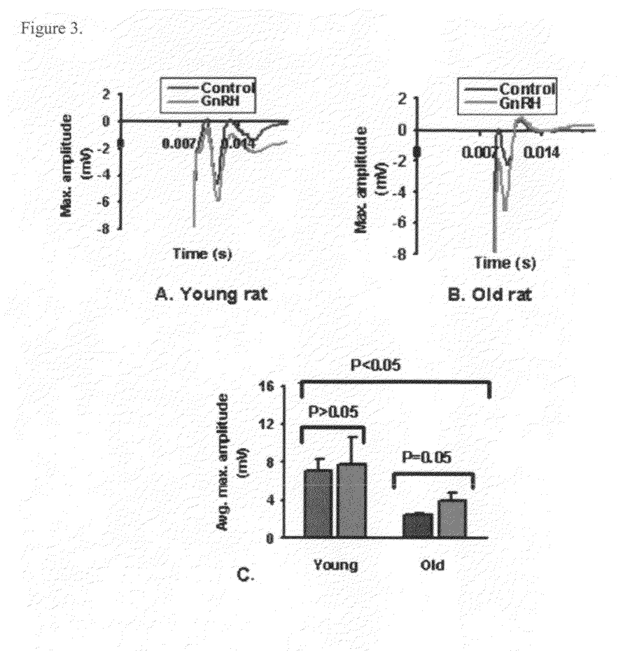

FIG. 3. GnRH1 increases neuronal excitability in hippocampal slices from old rats. Typical traces from hippocampal slice cut transversely at 500 μm thickness from 37 day (A) and 19 month (B) old male rats. Orthodromic extracellular field responses were recorded in the cell body layer of the CA1. Responses were evoked by a bipolar electrode with a constant current stimuli (50 μs duration) delivered every 30 s by electrodes placed in the schaeffer collaterals. 50 nM GnRH1 was used. Mean values are shown in C., n=5 slices each for young rats and n=3 slices each for old rats. Slices from two young and two old animals were used. Superfusion of human GnRH evoked a 50% increase in the maximum amplitude of population spikes in hippocampal slices from old rats compared to controls (FIGS. 3B, C). These results suggest that GnRH signaling via hippocampal GnRHR may be an important modulator of impulse transmission in the aging brain.

Heretofore, only a limited number of pharmacological agents have been identified as effective in treating symptoms of neurological disorders and neurodegenerative disease in a person suffering therefrom.

### DETAILED DESCRIPTION OF THE INVENTION

As indicated above, the invention entails treating neurological disorders or neurodegenerative disease in persons suffering therefrom and preventing neurological disorders or neurodegenerative disease in persons susceptible thereto.

A person “suffering from a neurological disorder or neurodegenerative disease” is a person who has been diagnosed as having a neurological disorders or neurodegenerative disease, by a practitioner of at least ordinary skill in the art of clinically diagnosing (e.g., diagnosing in patients antemortem) a neurological disorder or neurodegenerative disease, using methods and routines, such as those described above, that are standard in the art of such clinical diagnoses.

By “treating a neurological disorder or neurodegenerative disease” is meant slowing or preventing the progression or worsening of the disease that is now known to occur when the disorder or disease is untreated.

A person “susceptible” to a neurological disorder or neurodegenerative disease is a person who has not been diagnosed, by a practitioner of at least ordinary skill in clinical diagnosis of a neurological disorder or neurodegenerative disease, as suffering from the disorder or disease but for whom the probability of acquiring the disease, such that the person would be diagnosed as suffering from it, is significantly higher than for the general population. Thus, persons who are susceptible to AD include, for example, all persons who have not been diagnosed as definitely suffering from the disease but are suffering from Down's Syndrome due to trisomy 21, have been diagnosed by a practitioner of at least ordinary skill in clinical diagnosis of AD as possibly or probably but not definitely suffering from AD, have had head trauma, have a genetic marker for familial AD, such as one of those that are now known on chromosomes 1, 14, 19 and 21, or who are 80 years old or older. Persons who are susceptible to PD include, for example, all persons who have been diagnosed by a practitioner of at least ordinary skill in clinical diagnosis of PD as possibly or probably but not definitely suffering from PD, have had head trauma, have a genetic marker for familial PD, such as one of those that are now known on chromosomes 1, 2, 4, 6, 12 and X. Persons who are susceptible to stroke include, for example, all persons who have been diagnosed by a practitioner of at least ordinary skill in clinical diagnosis of stroke as possibly or probably but not definitely suffering from stroke, had brain trauma, had any one of a number of different cerebrovascular diseases such as hereditary cerebral hemorrhage with amyloidosis-Dutch type. Persons who are susceptible to neurodegeneration also include those you have sustained traumatic brain injury (TBI) from a closed head injury or a penetrating head injury.

By “preventing” a neurological disorder or neurodegenerative disease in a person susceptible thereto is meant preventing the development of the disorder or disease in such a person to the point that the person would be clinically diagnosed, by a practitioner of at least ordinary skill in the art of diagnosing a neurological disorder or neurodegenerative disease, as definitely suffering from a neurological disorder or neurodegenerative disease.

By “maintaining high progestagen levels” is meant elevating brain progestagen levels to levels comparable to pregnancy by administration of progestagens. Progestagens are lipid soluble and can be taken up into the brain following administration intramuscularly, intra-peritoneally, subcutaneously, intravenously, orally, topically, vaginally, rectally or via any other route that allows progestagens to enter the circulation of a person.

By administration of “progestagens” refers to any progesterone precursor, metabolite, analog or related compounds including but not limited to progesterone (pregn-4-ene-3,20-dione), pregnenolone ((3β)-3-hydroxypregn-5-en-20-one), pregnenolone sulfate sodium, 17α-hydroxypregnenolone (17α-hydroxypregn-4-en-20-yn-3-one), 17α-hydroxyprogesterone (17-hydroxypregn-4-ene-3,20-dione), allopregnenolone (allopregnan-3α-ol-20-one; 3α-hydroxy-5α-pregnan-20-one), alloprogesterone (5α-pregnane-3α-ol-20-one), levonorgestrel ((17α)-(±)-13-ethyl-17-hydroxy-18,19-dinorpregn-4-en-20-yn-3 -one), medroxyprogesterone ((6α)-17-hydroxy-6-methylpregn-4-ene-3,20-dione), etonogestrel ((17α)-13-Ethyl-17-hydroxy-11-methylene-18,19-dinorpregn-4-en-20-yn-3-one) or other progesterone analogs (including progestins).

By “other progesterone analogs” refers to but is not limited to (16α)-16,17-dihydroxypregn-4-ene-3,20-dione, [16α(R)]-16,17-[(1-phenylethylidene)bis(oxy)]pregn-4-ene-3,20-dione, 17-(acetyloxy)-6-chloropregna-4,6-diene-3,20-dione, (1α,2α)-6-chloro-1,2-dihydro-17-hydroxy-3′H-cyclopropa[1,2]pregna-1,4,6-triene-3,20-dione, 17-(acetyloxy)-6-chloropregna-1,4,6-triene-3,20-dione, 17-methyl-19-norpregna-4,9-diene-3,20-dione, 21-hydroxypregn-4-ene-3,20-dione, (9β,10α)-pregna-4,6-diene-3,20-dione, 17α-hydroxypregn-4-en-20-yn-3-one, 6α,11β,16α)-6-fluoro-11,21-dihydroxy-16,17-[(1-methylethylidene)bis(oxy)]pregn-4-ene-3,20-dione, (11β)-17-(acetyloxy)-9-fluoro-11-hydroxypregn-4-ene-3,20-dione, 17-[(1-oxohexyl)oxy]-19-norpregn-4-ene-3,20-dione, (11β)-11,17-dihydroxy-21-(4-methyl-1-piperazinyl)pregna-1,4-diene-3,20-dione, 6,17-dimethylpregna-4,6-diene-3,20-dione, (6α,11β)-11-hydroxy-6-methylpregn-4-ene-3,20-dione, 17-hydroxy-6-methylpregna-4,6-diene-3,20-dione acetate, 17-(acetyloxy)-6-methyl-16-methylenepregna-4,6-diene-3,20-dione, (17α)-17-hydroxy-19-norpregn-4-en-20-yn-3-one, (17α)-17-hydroxy-19-norpregn-5(10)-en-20-yn-3-one, 3-(cyclopentyloxy)-17-hydroxypregna-3,5-dien-20-one, (9β,10α)-6-chloropregna-1,4,6-triene-3,20-dione, (6α)-17-(acetyloxy)-6-(trifluoromethyl)pregn-4-ene-3,20-dione, 17-hydroxy-16-methylenepregna-4,6-diene-3,20-dione, Pregn-4-ene-3,11,20-trione, (3β)-21-(acetyloxy)-3-hydroxypregn-5-en-20-one, (17α)-(±)-13-ethyl-17-hydroxy-18,19-dinorpregn-4-en-20-yn-3-one, (7α,17β)-17-ethynyl-17-hydroxy-7-methylestr-5(10)en-3-one, (17α)-13-ethyl-11-methylene-18,19-dinorpregn-4-en-20-yn-17-ol, (2′S,6R,7R,8R,9S,10R,13S,14S,15S,16S)-1,3′,4′,6,7,8,9,10,11,12,13,14,15,16,20,21-Hexadecahydro-10,13-dimethylspiro[17H-dicyclopropa[6,7:15,16]cyclopenta[a]phenanthrene-17,2′(5′H)-furan]-3,5′(2H)-dione, ginsenosides.

By administration of “high-dose” progestagens is a dose of progestagens that elevates serum or brain progestagen levels above physiological concentrations to levels comparable to pregnancy.

By “promotes neurogenesis” refers to progestagens inducing the concomitant proliferation and differentiation of hESC into NPC/neural stem cells, or neural stem cells into specific neuronal cell types.

In accordance with the invention, a neurological disorder or neurodegenerative disease in a person suffering therefrom can be treated by administration to the person of any composition that increases the person's level of a hormone selected from the group consisting of progesterone, pregnenolone, pregnenolone sulfate sodium, 17α-hydroxypregnenolone, 17α-hydroxyprogesterone, allopregnenolone, alloprogesterone, levonorgestrel, medroxyprogesterone, etonogestrel or other progesterone analogs, in an amount and for a duration effective to bring about such an increase.

Further, in accordance with the invention, a neurological disorder or neurodegenerative disease can be prevented, or onset of clinical or behavioral manifestations delayed, in a person susceptible to the disease by administration to the person of any composition that increases the level of a hormone selected from the group of progestagens consisting but limited to progesterone, pregnenolone, pregnenolone sulfate sodium, 17α-hydroxypregnenolone, 17α-hydroxyprogesterone, allopregnenolone, alloprogesterone, levonorgestrel, medroxyprogesterone, etonogestrel or other progesterone analogs, in an amount and for a duration effective to bring about such an increase to a level above which development of the disease will not occur.

Reference herein to “level of a hormone” in a person means concentration of the biologically active hormone in the person's serum. Typically, the level of a hormone will be increased by increasing the concentration of the hormone itself. However, increasing the activity of the hormone—as, for example, by increasing the expression of PR with estrogens—even if the concentration of the hormone remains the same, is considered increasing the level of the hormone for purposes of the present application. The serum concentrations of biologically active progestagens in a human can be determined by any of a number of methods well known to the skilled. The serum concentrations of biologically active kisspeptin, GnRH, FSH, LH and inhibin in a human can be determined by any of a number of methods well known to the skilled.

As understood in the art, estrogens that stimulate the expression of the PR, can be employed to enhance the signaling of progestagens to neuronal cells. Thus, estrogens can be employed in accordance with the invention to directly increase the level of PR and thereby treat or prevent a neurological disorder or neurodegenerative disease. Estrogens can also therefore be given together with progestagens to enhance progestagen treatment or prevention of a neurological disorder or neurodegenerative disease.

As indicated above, progestagens (or physiologically acceptable salts thereof), such as micronized progesterone (Merck Index entry no. 07773) in oil delivered orally including Prometrium (U.S. Pat. No. 6,866,865), Utrogestan (U.S. Pat. No. 4,963,540), Minagest, and Microgest; or micronized progesterone gels delivered vaginally or rectally including Crinone (U.S. Pat. No. 5,543,150) and Prochieve (U.S. Pat. No. 5,543,150); or micronized progesterone in cocoa butter in the form of pessaries including Cyclogest; or progesterone tablets delivered vaginally including Endometrin; or NuvaRing delivered vaginally via a ring made of a flexible plastic (ethylene-vinyl acetate copolymer) can also be used in accordance with the invention to treat or prevent a neurological disorder or neurodegenerative disease by increasing levels of progestagens.

As indicated above, progestagens (or physiologically acceptable salts thereof), such as levonorgestrel delivered orally including Plan B; or vaginally including Mirena (U.S. Pat. No. 6,177,416) can also be used in accordance with the invention to treat or prevent a neurological disorder or neurodegenerative disease by increasing levels of progestagens.

As indicated above, progestagens (or physiologically acceptable salts thereof), such as medroxyprogesterone acetate delivered orally including Provera (U.S. Pat. No. 4,018,919); intramuscularly including Depo-Provera (U.S. Pat. No. 4,252,800); sub-cutaneously including Depo-SubQ Provera 104 (U.S. Pat. No. 4,252,800); or vaginally including NuvaRing (U.S. Pat. Nos. 5,262,408; 5,989,581; 6,544,546; 7,297,688; 7,323,454) can also be used in accordance with the invention to treat or prevent a neurological disorder or neurodegenerative disease by increasing levels of progestagens.

Preferred for use in the invention are progestagens and pharmaceutically acceptable salts thereof that can be employed to increase serum or brain progestagen to physiological or higher levels. Most preferred among these is etonogestrel given with or without estrogens (such as in NuvaRing) that releases a low dose of a progestin and an estrogen over a number of weeks, and, Mirena, Plan B and micronized oral progesterone preparations (Utrogestan, Minagest, and Microgest).

All of the U.S. patents cited herein, and all of the Merck Index entries cited herein are incorporated herein by reference.

Administration of progestagens in accordance with the invention will be by any method known in the art for administering same. Thus, administration may be oral, by injection subcutaneously, intramuscularly or intravenously of a sterile aqueous solution which includes the progestagen with buffers (e.g., sodium acetate, phosphate, sulphate), preservatives (e.g., benzy alcohol), salts (e.g., sodium chloride) and various excipients or carriers (e.g. oils, cocoa butter, polyethylene glycol). In this connection, see, for example, Physician's Desk Reference, 62.sup.st Ed., Thomson Healthcare, Montvale, N.J., U.S.A. (2007), pp. 3129-3134 (progesterone; Prometrium) and pp. 978-981 (progesterone gel; Crinone; Prochieve), which are also incorporated herein by reference.

The dose and dosage regimen for a particular composition used to carry out the invention with a particular patient will vary depending on the form of the progestagen (salt, micronized etc) and its concentration and other components in the composition, the route of administration, the gender, age, weight, and general medical condition of the patient, and whether the patient is already suffering from a neurological disorder or neurodegenerative disease. The skilled medical practitioner will be able to appropriately prescribe dosage regimens to carry out the invention. It is preferred in carrying out the invention that the concentrations of progestagens in a patient be increased to physiological levels or higher.

In carrying out the invention, compounds that increase progestagen signaling in the brain, or that stimulate production of progestagens, are administered at between about 5 □g and 400 mg per day.

In a most preferred embodiment of carrying out the invention, a composition comprising micronized progesterone will be administered intramuscularly or subcutaneously as a depot composition from which release of the analog into the patient's system will be sustained over a long period, from about a week to about six months or more. This will maintain the concentration progesterone in the patient at physiological or higher level(s) as described above without the pain, cost and inconvenience of much more frequent (e.g., daily) administration. Such depot compositions of progesterone are not known but their preparation is well within the skill of the ordinarily person skilled in the art. See, e.g., Physician's Desk Reference, 62.sup.st Ed. pp. 978-981 and 3335, cited above.

To allow the skilled medical practitioner to easily establish doses and dosage regimens of progestagens for treating or preventing a neurological disorder or neurodegenerative disease in individual patients in accordance with the invention, doses and dosage regimens for progestagens are provided here. Doses of progesterone effective to treat or prevent a neurological disorder or neurodegenerative disease range from about 10 μg to about 100 μg daily, preferably about 50 μg daily, with a 5 yearly subcutaneous injection of a sterile depot formulation of from about 50 mg to about 200 mg, preferably about 100 mg, with a subcutaneous injection once every five years of a sterile depot formulation. In the case or TBI, progesterone dosage is elevated to 1 mg daily for 3 months, and thereafter as described above.

Information from data already available or easily obtained by routine experimentation on progestagens in increasing progestagen signaling, those of ordinary skill can easily determine the dose and dosage regimens for any progestagen.

It must be noted that there would be no reason to use the methods of the invention for the purpose of treating or preventing a neurological disorder or neurodegenerative disease in a person who suffers from a condition whereby the person does produce progesterone at normal physiological levels or higher, with the exception of TBI.

For persons who do not produce progestagens at physiological levels, treatment in accordance with the invention must be continuous for the duration of their lives. The reason for this need for continuous administration is that, once such administration is discontinued, the persons' natural production of progesterone will be insufficient to prevent development of a neurological disorder or neurodegenerative disease.

Further, in accordance with the invention, a neurological disorder or neurodegenerative disease can be prevented, or onset of clinical or behavioral manifestations delayed, in a person susceptible to the disease by administration to the person of any composition that increases the level of a hormone selected from the group of progestagens together with any composition that reduces the person's level of a hormone selected from the group consisting of FSH and LH, increases the person's level of inhibin and modulates the person's level of GnRH in an amount and for a duration effective to bring about such an affect.

Further, in accordance with the invention, a neurological disorder or neurodegenerative disease can be prevented, or onset of clinical or behavioral manifestations delayed, in a person susceptible to the disease by administration to the person of any composition that increases the level of a hormone selected from the group of progestagens together with any composition that reduces the person's level of a hormone selected from the group consisting of FSH and LH, increases the person's level of inhibin and modulates the person's level of GnRH in an amount and for a duration effective to bring about such an affect where development of the disease will not occur.

In accordance with the invention, and as understood in the art, vaccines that stimulate production of antibodies can be employed to bind to kisspeptin, GnRH, FSH, or LH and block or at least substantially reduce their biological activities. Thus, vaccine-stimulated antibodies to kisspeptin, GnRH, FSH, or LH or more than one can be employed in accordance with the invention to directly reduce the level of these hormones and thereby treat or prevent neurological conditions and disease. Such antibodies to kisspeptin or GnRH, by blocking its activity, will result in reduced levels of FSH and LH. These antibodies can be employed in accordance with the invention to reduce levels of FSH and LH, or FSH or LH alone, and thereby to prevent or treat AD.

Antibodies for use in accordance with the invention may be made by conventional methods for preparation of vaccine antibodies for therapeutic use in humans. The vaccine-stimulated antibodies may be polyclonal and from any antibody-producing species, such as mice, rats, horses, dogs or humans. The antibodies may also be, and preferably are, monoclonal from cultures of antibody-producing cells from an antibody-producing species such as mice, rats, horses, dogs, and humans. The term “antibody” as used herein, unless otherwise limited, also encompasses antigen-binding fragments, such as F.sub.ab fragments, of intact antibodies. If an antibody is monoclonal but from cultured cells of a species other than human, the antibody may be “humanized” by conventional methods to make it more tolerable immunologically to a person treated therewith. Antibodies for use in accordance with the invention can also be made by conventional techniques using cultured cells, preferably human cells, that have been genetically engineered to make a desired intact antibody or antigen-binding antibody fragment.

Antibodies will be administered in accordance with the invention by any method known in the art for administering same but preferably by intravenous injection of a sterile aqueous solution of the antibody, together with standard buffers, preservatives, excipients and the like.

Also useful in carrying out the invention are compounds that agonize or antagonize the activity of GnRH or kisspeptin or stimulate production of inhibin. Thus, compounds that modulate the receptors for GnRH or kisspeptin, or stimulate receptors of inhibin on the pituitary, or that otherwise act on the pituitary-ovarian or pituitary-testicular axis to modulate production or activity of GnRH or kisspeptin or stimulate production of or activate inhibin, or to directly inhibit production of FSH or LH or both, will result in reduced levels of GnRH, FSH and LH and can be employed in accordance with the invention to treat or prevent neurological conditions and disease. One such compound is danazol (see The Merck Index, Merck & Co., Inc., Whitehouse Station, N.J., U.S.A 12.sup.th Ed. 1996 (hereinafter “Merck Index”), entry no. 2875, and U.S. Pat. No. 3,135,743). Such compounds, also, will be administered by any standard route as understood in the art.

As indicated above, GnRH or kisspeptin analogs (or physiologically acceptable salts thereof) and inhibin analogs (or physiologically acceptable salts thereof) can also be used together with progestagens in accordance with the invention to treat or prevent a neurological condition or disease by reducing levels of GnRH, kisspeptin, FSH and LH.

Preferred for use in the invention are GnRH analogs and pharmaceutically acceptable salts thereof that can be employed to reduce levels of FSH and LH to levels that are undetectable. Most preferred among these is leuprolide or goserelin, and especially leuprolide acetate and goserelin acetate.

By “other GnRH analogs” refers to but is not limited to agonists and antagonists of GnRH, FSH and LH such as leuprolide, buserelin nafarelin deslorelin histrelin goserelin triptorelin abarelix cetrorelix ganirelix acyline.

GnRH analogs or salts thereof that may be employed in accordance with the invention include, among others, GnRH itself and its monoacetate and diacetate salt hydrates (Merck Index entry no. 5500) and the many analogs thereof that are known in the art. These include, for example, leuprolide and its monoacetate salt (Merck Index entry no. 5484, U.S. Pat. No. 4,005,063); the analogs of leuprolide with the D-leucyl residue replaced with D-.α-aminobutyryl, D-isoleucyl, D-valyl or D-alanyl and the monoacetate salts thereof (U.S. Pat. No. 4,005,063); buserelin and its monoacetate salt (Merck Index entry no. 1527, U.S. Pat. No. 4,024,248); nafarelin and its monoacetate and acetate hydrate salts (Merck Index entry no. 6437, U.S. Pat. No. 4,234,571); deslorelin (Merck Index entry no. 2968); histrelin and its acetate salt (Merck Index entry no. 4760, U.S. Pat. No. 4,244,946); and goserelin and its acetate salt (Merck Index entry no. 4547, U.S. Pat. No. 4,100,274). For other GnRH analogs and salts thereof that can be used in accordance with the invention, see also U.S. Pat. No. 4,075,192, U.S. Pat. No. 4,762,717, and the U.S. patents cited at column 3, lines 49-54, of U.S. Pat. No. 4,762,717.

All of the U.S. patents cited herein, including those not cited specifically but cited at column 3, lines 49-54, of U.S. Pat. No. 4,762,717, and all of the Merck Index entries cited herein are incorporated herein by reference.

Administration of GnRH analogs, and kisspeptin analogs, in accordance with the invention will be by any method known in the art for administering same. Thus, administration may be by injection subcutaneously, intramuscularly or intravenously of a sterile aqueous solution which includes the analog together with buffers (e.g., sodium acetate, phosphate), preservatives (e.g., benzy alcohol), salts (e.g., sodium chloride) and possibly various excipients or carriers. In this connection, see, for example, Physician's Desk Reference, 51.sup.st Ed., Medical Economics Co., Montvale, N.J., U.S.A. (1997), pp. 2736-2746 (leuprolide acetate) and pp. 2976-2980 (goserelin acetate), which are also incorporated herein by reference.

The dose and dosage regimen for a particular composition used to carry out the invention with a particular patient will vary depending on the active (i.e., GnRH-, kisspeptin-, LH- or FSH-lowering) ingredient and its concentration and other components in the composition, the route of administration, the gender, age, weight, and general medical condition of the patient, and whether the patient is already suffering from AD. The skilled medical practitioner will be able to appropriately prescribe dosage regimens to carry out the invention. It is preferred in carrying out the invention that the concentrations of GnRH, kisspeptin, FSH or LH, preferably both, in a patient be reduced to and maintained at levels that are as low as possible. It is usually preferred that the concentrations of FSH and LH be reduced to indetectable levels. However, beneficial effects of preventing or reducing susceptibility to AD, or treating AD, are achieved even if the concentrations of FSH and LH are reduced but not to indetectable levels. Thus, the medical practitioner will select the composition, dose and dosage regimen for a particular patient to achieve and maintain such low concentrations of GnRH, FSH and/or LH in the patient.

In carrying out the invention, compounds that block the receptors for kisspeptin on the hypothalamus, GnRH on the pituitary or stimulate receptors of inhibin on the gonads, or that otherwise act on the hypothalamic-pituitary-ovarian or hypothalamic-pituitary-testicular axis to inhibit production or activity of kisspeptin or GnRH or stimulate production of or activate inhibin, or to directly inhibit production of FSH or LH or both, are administered at between about 0.1 g and 10 g per day.

In a most preferred embodiment of carrying out the invention, a composition comprising a GnRH analog will be administered intramuscularly or subcutaneously as a depot composition from which release of the analog into the patient's system will be sustained over a long period, from about a week to about six months or more. This will maintain the concentration of GnRH, FSH and/or LH in the patient at the low or undetectable level(s) as described above without the pain, cost and inconvenience of much more frequent (e.g., daily) administration. Such depot compositions of GnRH analogs are known and their preparation is well within the skill of the ordinarily person skilled in the art. See, e.g., Physician's Desk Reference, 51 .sup.st Ed. pp. 2736-2746 and 2976-2980, cited above.

To allow the skilled medical practitioner to easily establish doses and dosage regimens of GnRH analogs for treating or preventing neurological disorders or neurodegenerative diseases in individual patients in accordance with the invention, doses and dosage regimens for goserelin acetate and leuprolide acetate are provided here. Doses of goserelin acetate effective to treat or prevent neurological disorders or neurodegenerative diseases range from about 1 mg to about 10 mg, preferably about 4 mg, with a once monthly subcutaneous injection of a sterile depot formulation of from about 3 mg to about 30 mg, preferably about 10 mg, with a subcutaneous injection once every three months of a sterile depot formulation.

Doses of leuprolide acetate effective to treat or prevent neurological disorders or neurodegenerative diseases range between about 0.2 and 20 mg/day, preferably about 1 mg/day, when the dosage regimen is by once daily, subcutaneous injection of sterile solution comprising the compound; between about 1 mg and about 10 mg, preferably about 5 mg, with a once monthly intramuscular injection of a sterile depot formulation comprising the compound; and between about 10 mg and about 50 mg, preferably about 25 mg, with an intramuscular injection once every three months of a depot formulation comprising the compound.

Information from data already available or easily obtained by routine experimentation on GnRH analogs in suppressing GnRH, LH and FSH activity, those of ordinary skill can easily determine the dose and dosage regimens for any GnRH analog.

It must be noted that there would be no reason to use the methods of the invention for the purpose of treating or preventing neurological disorders or neurodegenerative diseases in a person who suffers from a condition whereby the person either does not produce GnRH or kisspeptin (and so does not produce LH or FSH). The levels of GnRH, kisspeptin, LH and FSH cannot be reduced in such a person by administering compounds in accordance with the method of the invention; and, as the skilled will understand, in the case of administering GnRH analogs to such a person, the levels of LH and FSH in the person might increase under certain circumstances.

For persons who do produce GnRH, kisspeptin, FSH and/or LH, treatment in accordance with the invention must be continuous for the duration of their lives. The reason for this need for continuous administration is that, once such administration is discontinued, the persons' natural production of GnRH, FSH and LH will resume within at most a few months or, more typically, within a few weeks.

By “embryonic stem cells” is meant cells that are pluripotent or totipotent.

By “embryoid bodies” is meant a spherical cystic structure that contains the 3 germ cell layers (endoderm, mesoderm and ectoderm).

By “rosettes” is meant neuroectodermal cells in a columnar rosette structure containing NPC.

By “primary neurons” is meant any neuronal cell type that has been removed from either an embryonic, fetal or post-natal animal.

A compound that “blocks PR, ER or OR signaling” includes any agent that blocks signaling via PR, ER or OR including but not limited to antagonists, agonists, antibodies against the receptor or other compounds that decrease or inhibit signaling via these receptor pathways.

By “sufficient time” refers to the time that it takes to generate either EBs or rosettes.

Are “grown in media containing essential nutrients” refers to a media containing buffers and salts at physiologically appropriate pH that includes vitamins, minerals, metabolic precursors such as amino acids and monosaccharides, and growth factors required to maintain hESC, EBs or rosettes.

By “inhibiting blastulation” means preventing the formation of blastulas in vivo, or preventing the differentiation of hESC into EBs containing the 3 germ cell layers in vitro.

By “inhibiting neurogenesis” means preventing differentiation of hESC or EBs into neuronal cell types.

The foregoing description, discussion and scope of the invention are directed to those of ordinary skill in the treatment of actual or incipient neurological disorder or neurodegenerative disease, or the performance of experiments. Accordingly, it is to be expected that the teachings herein will enable selection of specific agents and regimens for treatment within the scope of the appended claims. It is also expected that the teaching herein will enable selection of specific agents for promoting or inhibiting neurogenesis.

### REFERENCES

Bäckman, L., Small, B. J., Wahlin, A., Larsson, M. (1999). Cognitive functioning in very old age. In: Craik, F. I. M., Salthouse, T. A. (eds) Handbook of cognitive aging, Hillsdale, N.J.: Erlbaum, vol 2, pp499-558.
Bowen, R. L., Smith, M. A., Harris, P. L. R., Kubat, Z., Martins R N, Castellani, R. J., Perry, G. and Atwood, C. S. (2002). Elevated luteinizing hormone expression colocalizes with neurofibrillary tangles in Alzheimer disease. Journal of Neuroscience Research, 70(3), 514-518.
Bowen, R. L., Verdile, G., Liu, T., Perry, G., Smith, M. A., Martins, R. N. and Atwood, C. S. (2004). Luteinizing hormone, a reproductive regulator that modulates the processing of amyloid-β protein precursor and amyloid-β deposition. Journal of Biological Chemistry, 279, 20539-45.
Bowen R. L. and Atwood C. S. (2004). Living and dying for sex. A theory of aging based on the modulation of cell cycle signaling by reproductive hormones. Gerontology, 50, 265-290.
Casadesus, G., Webber, K. M., Atwood, C. S., Pappolla, M. A., Perry, G., Bowen, R. L. and Smith, M. A. (2006a). Luteinizing hormone modulates cognition and amyloid-β deposition in Alzheimer APP transgenic mice. Biochemica Biophyica. Acta 1762, 447-452.
Casadesus, G., Webber, K. M., Atwood, C. S., Bowen, R. L., Perry, G., Smith, M. A. (2006b). Luteinizing hormone mediates Alzheimer-type changes in neurons. 10th International Conference on Alzheimer's Disease and Related Disorders, P3-296.
Casadesus, G., Milliken, E. L., Webber, K. M., Bowen, R. L., Lei, Z., Rao, C. V., Perry, G., Keri, R. A., Smith, M. A. (2007). Increases in luteinizing hormone are associated with declines in cognitive performance. Mol Cell Endocrinol.269, 107-111.
De la Torre, J. C. (1997) Hemodynamic consequences of deformed microvessels in the brain in Alzheimer's disease. Ann. N. Y. Acad. Sci. 826, 75-91.
Evans, D. A., Funkenstein, H. H., Albert, M. S., Scherr, P. A., Cook, N. R., Chown, M. J., Hebert L. E., Hennekens C. H., and Taylor J. O. (1989) Prevalence of Alzheimer's disease in a community population of older persons. Higher than previously reported, JAMA 262, 2551-2556.
Glabe, C. G. (2006) Common mechanisms of amyloid oligomer pathogenesis in degenerative disease. Neurobiol Aging 27 570-575.
Hardy, J., Selkoe, D. J. (2002). The amyloid hypothesis of Alzheimer's disease: progress and problems on the road to therapeutics. Science, 297, 353-356.
Hebert, L. E., Scherr, P. A., Bienias, J. L., Bennett D. A. and Evans D. A. (2003) Alzheimer disease in the US population: prevalence estimates using the 2000 census, Arch Neurol 60, 1119-1122.
Hoge, C. W., McGurk, D., Thomas, D. L., Cox, A. L., Engel, C. C., Castro, C. A. (2008) Mild traumatic brain injury in U.S. soldiers returning from Iraq. The New England Journal of Medicine 358, 453-463.
Jellinger, K. A. (2002) Recent developments in the pathology of Parkinson's disease. J. Neural Transm. Suppl. 62, 347-376.
Kalaria, R. N. (1996). Cerebral vessels in ageing and Alzheimer's disease. Pharmacol. Ther. 72, 193-214.
Klein, W. L. (2002). Abeta toxicity in Alzheimer's disease: globular oligomers (ADDLs) as new vaccine and drug targets, Neurochem Int 41, 345-352.
Levine, R. M., Rubalcaba, E., Lippman, M. E., Cowan, K. H. (1985) Effects of estrogen and tamoxifen on the regulation of dihydrofolate reductase gene expression in a human breast cancer cell line. Cancer Res 45, 1644-50
Petersen, R. C. and Morris, J. C. (2003) Clinical features., in: Mild Cognitive Impairment, R. C. Petersen and. eds., Oxford University, New York, pp. 15-39.
Raina, A. K., Zhu, X., Rottkamp, C. A., Monteiro, M., Takeda, A. Smith M A. (2000). Cyclin' toward dementia: cell cycle abnormalities and abortive oncogenesis in Alzheimer disease. J. Neurosci. Res. 61, 128-133.
Ross, J. S., Stagliano, N. E., Donovan, M. J., Breitbart, R. E. and Ginsburg, G. S. (2001). Atherosclerosis: a cancer of the blood vessels? Am. J. Clin. Pathol. 116, S97-107.
Traumatic Brain Injury: Hope Through Research. NINDS. Publication date February 2002. NIH Publication No. 02-2478. Prepared by: Office of Communications and Public Liaison, National Institute of Neurological Disorders and Stroke, National Institutes of Health
Vadakkadath Meethal, S. and Atwood, C. S. (2005). Alzheimer's Disease: The Impact of Age-related Changes in Reproductive Hormones. The Role of Hypothalamic-Pituitary-Gonadal Hormones in the Normal Structure and Functioning of the Brain, Cellular and Molecular Life Sciences, 62, 257-270.
Yang, Y., Geldmacher, D. S., Herrup, K. (2001). DNA replication precedes neuronal cell death in Alzheimer's disease. J Neurosci. 21, 2661-2668.

What is claimed is:

1. A method for treating or preventing a neurological disorder or neurodegenerative disease in a human subject in need thereof by administering to said subject a therapeutically effective amount of at least one physiologically acceptable agent, that increases blood serum and brain levels of one or more progestagen (progesterone, pregnenolone, 17α-hydroxypregnenolone, 17α-hydroxyprogesterone, allopregnenolone, alloprogesterone, levonorgestrel, medroxyprogesterone, etonogestrel or other progesterone analogs).

2. The method of claim 1 in which said agent increases progestagen, is formulated in a pharmaceutically acceptable carrier or diluent such as a pill for oral administration, gel for cutaneous/dermal or vaginal administration, or sustained-release composition for suppository, or intramuscular, intravenous or subcutaneous injection.

3. The method of claim 1 in which said agent is administered at a dose to elevate progestagen to physiological levels.

4. The method of claim 1 in which said agent is administered in high dosage comparable to pregnancy levels.

5. A method for treating or preventing a neurological disorder or neurodegenerative disease in a human subject in need thereof by promoting neurogenesis by administering to said subject a therapeutically effective amount of at least one physiologically acceptable agent that increases blood serum and brain levels of one or more progestagen (progesterone, pregnenolone, 17α-hydroxypregnenolone, 17α-hydroxyprogesterone, allopregnenolone, alloprogesterone, levonorgestrel, medroxyprogesterone, etonogestrel or other progesterone analogs).

6. The method of claim 5 in which a therapeutically effective amount of estrogen is administered together with a progestagen.

7. A method for treating or preventing a neurological disorder or neurodegenerative disease in a human subject in need thereof by administering to said subject a therapeutically effective amount of at least one physiologically acceptable agent, that increases blood serum and brain levels of one or more progestagen plus decreases blood serum and brain levels of one or more of kisspeptin, GnRH, FSH and LH.

8. The method of claim 7 where said agent is (1) kisspeptin, GnRH, inhibin or analogs thereof, (2) anti-kisspeptin, anti-GnRH, anti-inhibin antibodies, anti-FSH and/or anti-LH antibodies, (3) RNAi or antisense to kisspeptin, GnRH, inhibin, FSH and/or LH.

9. The method of claim 7 where said agent is leuprolide, buserelin nafarelin deslorelin histrelin goserelin triptorelin abarelix cetrorelix ganirelix acyline or physiologically acceptable analogs and salts thereof.

10. The method of claims 1 and 9 in which the neurological disorder or neurodegenerative disease is selected from the group consisting of Alzheimer's disease; fronto-temporal dementia; cerebrovascular disease; stroke; Parkinson's disease; amyotrophic lateral sclerosis; multiple sclerosis; central or peripheral nervous system damage, dysfunction, or complications involving same stemming from edema, injury, or trauma; neurodegenerative changes in postmenopausal women and andropausal men; carpel tunnel syndrome; Charcot-Marie-Tooth disease; diabetic neuropathy; neurofibromatosis; peripheral neuropathy; prion diseases; progressive supranuclear palsy; restless leg syndrome; spinal cord injury; tardive dyskinesia; brain tumors; and neurological developmental disorders including autism, Angelman syndrome and cerebral palsy.

11. The method of claims 1 and 9 in which said neurological disorder is an andropausal or postmenopausal disorder.

12. A method for inhibiting blastulation during embryogenesis in a human or animal by administering to said subject an effective amount of at least one agent that blocks progesterone and/or estrogen signaling, where said agents are progesterone receptor (PR) antagonist including but not limited to RU-486; estrogen receptor (ER) antagonists including but limited to ICI 182,780 and/or opioid receptor (OR) antagonists including but not limited to ICI 174,864.

13. The method of claim 12 in which said agents that decrease PR, ER or OR signaling, is formulated in a pharmaceutically acceptable carrier or diluent such as a pill for oral administration, gel for cutaneous or vaginal administration, or sustained-release composition for suppository, or intramuscular, intravenous or subcutaneous injection.

14. The method of claim 12 in which said agent is administered at a dose to suppress PR, ER or OR signaling below physiological levels, preferably to low levels.

15. A method for inhibiting neurogenesis during embryogenesis, fetal, neonatal, childhood, puberty or adult life in a human or animal by administering to said subject an effective amount of at least one agent that blocks PR, ER and OR signaling, where said agents are PR antagonist including but not limited to RU-486; ER antagonists including but limited to ICI 182,780 and/or OR antagonists including but not limited to ICI 174,864.

16. The method of claim 15 in which inhibition of neurogenesis is utilized during in vitro fertilization techniques, or in a neurological disorder selected from the group consisting of brain cancer, autism, Angelman syndrome and cerebral palsy.

17. The method of claim 15 in which said agents that decrease PR, ER or OR signaling, is formulated in a pharmaceutically acceptable carrier or diluent such as a pill for oral administration, gel for cutaneous or vaginal administration, or sustained-release composition for suppository, or intramuscular, intravenous or subcutaneous injection.

18. The method of claim 15 in which said agent is administered at a dose to suppress PR, ER or OR signaling below physiological levels.

19. A method for inducing neurogenesis in vitro where embryonic stem cells (ESC) are grown in media containing essential nutrients plus progestagen for sufficient time to generate neuronal cell types.

20. The method of claim 19 where the cell types are human or animal ESC, embryoid bodies (EBs), rosettes or primary neurons from embryos or adult brains.

21. The method of claim 19 where the media contains an effective level of one or more progestagen (progesterone, pregnenolone, 17α-hydroxypregnenolone, 17α-hydroxyprogesterone, allopregnenolone, alloprogesterone, levonorgestrel, medroxyprogesterone, etonogestrel or other progesterone analogs).

22. The method of claim 19 in which an estrogen is administered together with a progestagen.
